(19) United States
(12) Patent Application Publication
(10) Pub. No.: US 2005/0176111 A1
(43) Pub. Date: Aug. 11, 2005

(54) Directable exhaust for water sport tow boat

(76) Inventors: Richard D. Lee, Incline Village, NV (US); Timothy M. Lopes, Merced, CA (US)

Correspondence Address: James W Paul;Fulwider Patton Lee & Utecht, Howard Hughes Center, 6060 Center Drive Tenth Floor, Los Angeles, CA, 90045, US

(21) Appl. No.: 10/491,740

(22) PCT Filed: Oct. 29, 2003
(86) PCT No.: PCT/US03/34590

Related U.S. Application Data
(63) Continuation-in-part of application No. 10/608,787, filed on Jun. 26, 2003.
(60) Provisional application No. 60/422,351, filed on Oct. 30, 2002.

(51) Int. Cl. C12P 19/12

(52) U.S. Cl. 435/100

(57) ABSTRACT

The directable exhaust for a wake surfing power boat permits directing exhaust out of either the port or starboard side of a boat opposite the wake wave on which a wake surfer is surfing to avoid exposure of the person to the exhaust fumes. Valves which direct the exhaust out of the side of the boat are linked such that the exhaust can be directed out of either the port or starboard side of the boat away from the wake surfer by a single control available to the boat operator.

### RELATED APPLICATIONS

This is a continuation in part of Ser. No. 10/608,787, filed Jun. 26, 2003, which is based on Provisional Application No. 60/422,351, filed Oct. 30, 2002.

### BACKGROUND OF THE INVENTION

1. Field of the Invention

This invention relates generally to an apparatus and method for exhausting fumes of a power motor boat, and more particularly concerns an apparatus and method for directing exhaust fumes of a-power motor boat away from a person wake surfing behind the power motor boat to reduce exposure of the person to the exhaust fumes.

2. Description of Related Art

Wake surfing behind power boats has become a popular sport, and in response, manufacturers of such power boats used for wake surfing have created new configurations of boats which enhance the size of the waves produced behind and to the port and starboard sides of the power boats to improve the sport and the opportunities for more spectacular surfing on lakes and rivers. In wake surfing, a rider on a surfboard is towed in a standing position behind a power boat. At a speed of about 10-14 mph, the wake of the boat is large enough to allow the surfer to ride the wake with little or no tension on the tow rope, simulating ocean surfing. Wake surfing can be performed on either the right or left outside faces of the boat wakes, approximately 8 to 20 feet behind the transom of the boat.

As wake surfing has become more popular, certain limitations on existing boats used for the purpose have been identified. One such limitation is that most boats used for the purpose have exhaust systems which direct the exhaust out of the back of the boat, either above or below the water line, thus causing the wake surfer behind the boat and to either the port or starboard side to be exposed to exhaust fumes and carbon monoxide contained in the exhaust, thus diminishing the enjoyment and safety of the sport.

Ambient carbon monoxide concentrations as high as 27,000 parts per million (ppm) have been measured at the stern of power boats involved in boating fatalities attributed to carbon monoxide. The U.S. National Park Service and U.S. Coast Guard have recently documented a number of fatal and non-fatal carbon monoxide poisonings resulting from exposure to engine exhaust from a variety of boat types, most of which occurred while on or near a swim platform or seated in the stern of the boat. The U.S. Coast Guard consequently issued a safety alert in 2001 advising boaters not to “engage in the dangerous and illegal practice teak surfing,” in which a person holds on to a swim platform while a wake forms, and then trails behind the boat while holding on to the swim step.

Although power boat exhaust valves are known that can be used to direct engine exhaust through the transom or stern of a boat, these are typically designed to prevent exhaust fumes from entering the deck area of a power boat, or for purposes of sound reduction, and none of these systems reduce the carbon monoxide exhausted behind the boat and to either the port or starboard side of a 1 power boat where a person would wake surf. It would therefore be desirable to provide an apparatus for substantially reducing the presence of exhaust fumes behind and/or to one side of a power boat, and for substantially reducing the danger to a person behind and to one side of the power boat, such as a wake surfer trailing behind such a power boat, from exposure to exhaust fumes, and particularly carbon monoxide, from such a power boat. The present invention meets these and other needs.

### SUMMARY OF THE INVENTION

Briefly and in general terms, the present invention provides for an apparatus and method for directing exhaust out of either the port or starboard side of a boat, either above or below the water line of the boat, away from a person behind and to either the port or starboard side of the power motor boat to avoid exposure of the person to the exhaust fumes. In a presently preferred alternative embodiment, the exhaust may be directed vertically to exhaust the fumes up and away from a wake surfer in a wave directly behind the boat. In the case where the person behind the power boat is wake surfing, the apparatus permits the engine exhaust to be directed to either side of the boat, either above or below the water line of the boat, away from the wake wave on which the wake surfer is surfing, thus diminishing the amount of exhaust fumes to which the surfer is exposed. The present invention can be applied to inboard/outdrive systems and outboard systems as well.

According to the invention, valves which direct the exhaust out of the side of the boat are controlled such that the exhaust can be directed to either the port or starboard side of the boat away from the wake surfer by a single control available to a boat operator or observer on the boat. The exhaust can be an underwater exhaust or an above-water exhaust. In one presently preferred configuration, dual valves in the exhaust system are connected to an engine exhaust manifold such that directing the exhaust to the starboard side of the boat closes off the flow of exhaust to the port side of the boat, and directing the exhaust to the port side of the boat closes off the flow of exhaust to the starboard side of the boat. In one aspect, the flow of exhaust from one side of the boat to the other is switched by a control unit. In another preferred aspect, starboard and port exhaust pipes may be connected to the engine exhaust manifold at about a 90 degree angle. The coordinated operation of the dual valves may be linked by an electrical control mechanism such that the valves will direct exhaust to one side or the other on the basis of a single electrical switch located conveniently to the operator or an observer in the boat. In another embodiment, linkage of the valves may be mechanical in nature, and the valve mechanisms may be linked to a single mechanical control such as a control cable located in the cockpit or convenient to an observer in the boat.

In an alternative embodiment, the system of the invention can also be used to divert exhaust in a vertical direction, above the surface of the water, a distance sufficient to reduce the exhaust which trails behind the boat at the water level. In such an embodiment, the diversion valves may either direct the exhaust to one side or the other as described above, or divert the exhaust to a vertical exhaust pipe extruding a desired distance above the boat.

By use of the invention, wake surfing is substantially enhanced and any danger associated with exposing the wake surfer to exhaust fumes is substantially diminished.

Other features and advantages of the present invention will become more apparent from the following detailed description of the preferred embodiments in conjunction with the accompanying drawing, which illustrates, by way of example, the operation of the invention.

### BRIEF DESCRIPTION OF THE DRAWING

FIG. 1 is a schematic diagram of a first embodiment of an apparatus for directing a boat's engine exhaust out of either the port or starboard side of the boat, according to the invention.

FIG. 2 is a schematic diagram of an alternate embodiment of an apparatus, in which the muffler is outside the transom of the boat, for directing a boat's engine exhaust out of either the port or starboard side of the boat, according to the invention.

### DETAILED DESCRIPTION OF THE PREFERRED EMBODIMENTS

While power boat exhaust valves commonly direct engine exhaust through the transom or back end of a boat, this typically does not reduce carbon monoxide levels from boat engine exhaust behind and/or to one side of the boat.

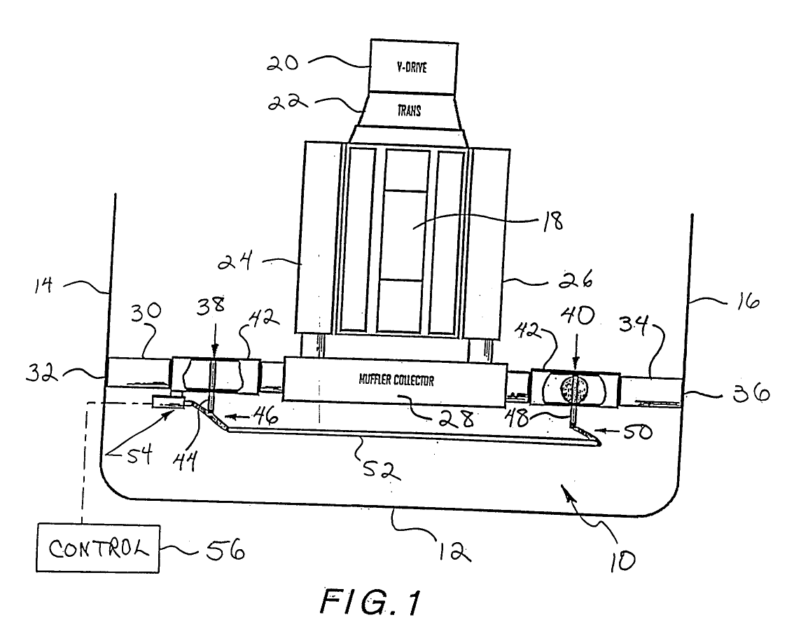

Referring to FIG. 1, the invention provides for a directable exhaust system 10 for directing a engine exhaust of a boat (not shown) out of either the port or starboard side of the boat, either above or below a water line of the boat (not shown), and away from a person behind the boat, such as a wake surfer, for example, to reduce exposure of the person to the engine exhaust and potentially dangerous carbon monoxide fumes. The boat typically has a transom at the aft end or stern 12, a port side 14, and a starboard side 16. The boat is typically a power motor boat having an engine 18, a V-drive 20, a transmission 22, and at least one exhaust manifold, such as port and starboard exhaust manifolds 24, 26. The exhaust manifolds may include a muffler collector 28 connected to the exhaust manifolds to receive the exhaust from the exhaust manifolds, and a first exhaust conduit 30, having a port side exhaust outlet 32, is connected to the muffler collector of the exhaust manifolds. A second exhaust conduit 34, having a port side exhaust outlet 36, is similarly connected to the muffler collector. In one aspect, the first and second exhaust conduits are typically connected by the muffler collector to the engine exhaust manifolds at about a 90 degree angle.

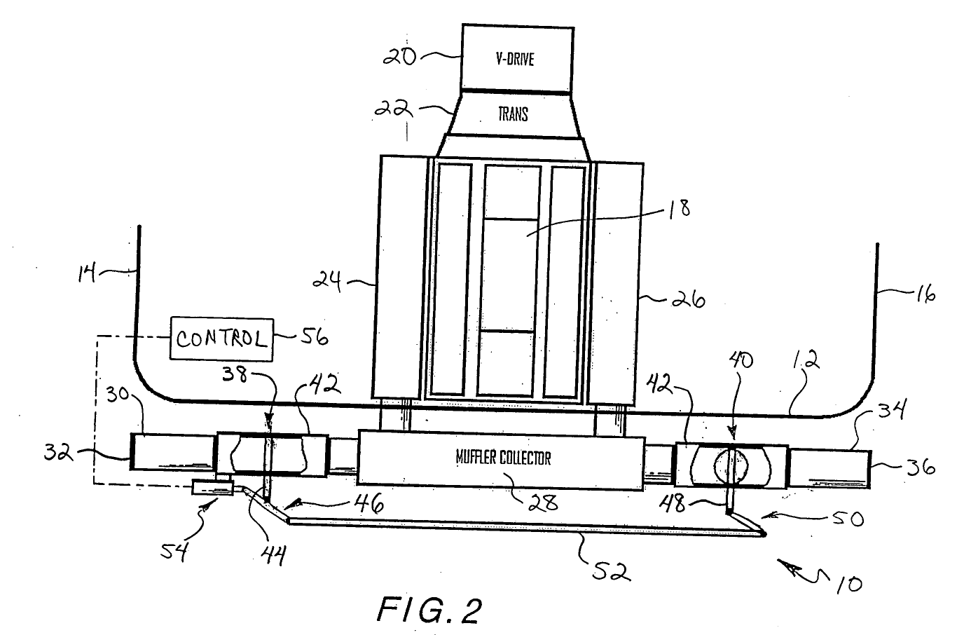

Although FIG. 1 shows the muffler 28 as being located forward of the transom, with the valves and exhaust ports located internally in the boat, in an alternate embodiment illustrated in FIG. 2, in which elements identical to those in FIG. 1 are identified like reference numerals, the muffler 28 may be located outside the transom, with the valves and exhaust ports located external to the boat. Such an external system advantageously allows the system of the invention to be readily added to pre-existing motor boat exhaust systems.

Valve means are provided for directing the engine exhaust out of one of the port and starboard exhaust outlets, such as a port butterfly valve 38 connected to the first exhaust conduit for controlling flow of engine exhaust through the first exhaust conduit to the port exhaust outlet, and a starboard butterfly valve 40 connected to the second exhaust conduit for controlling flow of engine exhaust through the second exhaust conduit to the starboard exhaust outlet, each valve having a valve housing 42. The port butterfly valve includes a port valve shaft 44, having a port valve shaft arm 46, and the starboard butterfly valve includes a starboard valve shaft 48 having a starboard valve shaft arm 50. Means for linking the first and second valves are provided, such as a connector rod 52 which can be connected to each of the valve shaft arms, such that opening of the starboard butterfly valve closes the port butterfly valve, as is illustrated, and opening the port butterfly valve closes the starboard butterfly valve. In this manner the engine exhaust is directed out of one of the port and starboard side exhaust outlets but not the other. The action of the connector rod linkage in controlling the coordinated operation of the valves can be controlled by an actuator motor, such as an electric actuator motor 54, which may be connected to a controller unit 56 with an electrical switch (not shown) that can be located in the boat in a place convenient for the boat operator or an observer, such as on the deck of the boat. Either alternatively or in addition to the electrical control of the coordinated operation of the valves by control of the actuator motor, a mechanical control of the connector rod connected between the first and second valves may be provided, as for example a control cable, such as a control cable available under the trade name “MORSE CABLE” from Morse Controls Ltd. of the United Kingdom, for opening one of the valves and closing the other of the valves. The mechanical control can be located in the cockpit of the boat convenient to an operator of the boat, for example, or otherwise may be located elsewhere convenient to an observer in the boat. Alternatively, the valves may be electrically actuated individually, operating under a coordinated control system such that opening one valve closes the other. The system may also be operated automatically by a wind sensing control mechanism (not shown), to send the exhaust to an appropriate downwind side of the boat, away from occupants of the boat.

While the invention has been described in terms of an apparatus for directing exhaust gases to one side or the other for an inboard power boat, the system described may be used with slight modification to provide the same benefits for inboard/outdrive systems in which the engine and outdrive are a unit with the engine in the boat connected to an outdrive propulsion unit of either the propeller or jet drive type. It will also be appreciated by those skilled in the art that the apparatus of the invention can be used to direct exhaust in directions other than to one side or the other, such as vertically, to reduce the exhaust fumes in the wake of the boat.

It should be recognized that the invention may also be applied to other types of motorized boats, such as houseboats, sport fishing boats and pleasure craft, for example, so that exhaust can be routed to a side of the boat that would allow the exhaust fumes to be sent downwind away from occupants of the boat. The system could be operated automatically by a wind sensing control mechanism to send the exhaust to the appropriate port or ports.

### EXAMPLE

### Power Boat Exhaust System Carbon Monoxide Test

Standard Exhaust

Carbon monoxide levels were measured behind a Centurion Avalanche test boat with standard transom exhaust ports, with a Thermo Gas Tech Screamer™ personal portable carbon monoxide monitor at about 2 PM at the Fineline Merced factory Test Lake, with light cross winds from approximately the southeast at about 0 to 5 mph. Air samples were taken approximately 5 inches behind the boat and 4 inches off the water. With the boat path northwest with the wind, the peak carbon monoxide level was found to be 250 ppm. With the boat path southeast into the wind, the peak carbon monoxide level was found to be 130 ppm. Both of these measured levels were well above the World Health Organization's recommended maximum carbon monoxide level of 87 ppm.

Side Draft Exhaust

Carbon monoxide levels were measured behind a Centurion prototype Cyclone test boat with side draft exhaust ports out the starboard side, with the same portable carbon monoxide monitor at about 10 AM at the Fineline Merced factory Test Lake, with light cross winds from approximately the southeast again at about 0 to 5 mph. Air samples were taken approximately 5 inches behind the boat on the left side or port side of the wake and 4 inches off the water. With the boat path northwest with the wind, the peak carbon monoxide level was found to be 14 ppm. With the boat path southeast into the wind, the peak carbon monoxide level was found to be 0 ppm. Both of these measured levels were well below the World Health Organization's recommended maximum carbon monoxide level of 87 ppm. These results demonstrate a significant reduction in the carbon monoxide levels, and demonstrate that the switching of exhaust to one of the port and starboard sides of a boat according to the invention can have a significant effect in reducing carbon monoxide levels from boat engine exhaust behind and on the other side of boat.

It will be apparent from the foregoing that while particular forms of the invention have been illustrated and described, various modifications can be made without departing from the spirit and scope of the invention. Accordingly, it is not intended that the invention be limited, except as by the appended claims

1. An apparatus and method for directing a boat's engine exhaust away from a person behind the boat to reduce exposure of the person to the engine exhaust, the boat having a engine with at least one exhaust manifold, the apparatus comprising:
a port side exhaust outlet connected by a first exhaust conduit to the at least one engine exhaust manifold of the boat;
a starboard side exhaust outlet connected by a second exhaust conduit to the at least one engine exhaust manifold of the boat; and
valve means connected to said first and second exhaust conduits for directing the engine exhaust to one of the port and starboard exhaust outlets.

2. The apparatus of claim 1, wherein said first and second exhaust conduits are connected to the at least one engine exhaust manifold at about a 90 degree angle.

3. The apparatus of claim 1, wherein the valve means comprises a first valve connected to the first exhaust conduit for controlling flow of engine exhaust through the first exhaust conduit to the port side exhaust outlet, and a second valve connected to the second exhaust conduit for controlling flow of engine exhaust through the second exhaust conduit to the starboard side exhaust outlet, and means for linking said first and second valves so that opening of one of the first and second valves closes the other of the first and second valves, such that the engine exhaust is directed out of one of the port or starboard side exhaust outlets.

4. The apparatus of claim 3, wherein the first and second valves are electrically controlled valves, and the means for linking the first and second valves comprises an electrical switch for opening one of said first and second valves and closing the other of said first and second valves.

5. The apparatus of claim 3, wherein the means for linking comprises a mechanical linkage between said first and second valves.

6. The apparatus of claim 1, wherein the valve means comprises a first valve connected to the first exhaust conduit for controlling flow of engine exhaust through the first exhaust conduit to the port side exhaust outlet, and a second valve connected to the second exhaust conduit for controlling flow of engine exhaust through the second exhaust conduit to the starboard side exhaust outlet, and means for linking said first and second valves so that opening of one of the first and second valves closes the other of the first and second valves, such that the engine exhaust is directed out of one of the port or starboard side exhaust outlets, and further comprising valve control means for controlling the valve means to switch the flow of engine exhaust from one of said port and starboard side exhaust outlets to the other of the port and starboard-side exhaust outlets.

7. The apparatus of claim 6, wherein the means for linking comprises a mechanical linkage between said first and second valves, and said valve control means comprises a mechanical control connected to said mechanical linkage for opening one of said first and second-valves and closing the other of said first and second valves.

8. The apparatus of claim 1, wherein the valve means is located outside the boat.

9. The apparatus of claim 1, wherein the port side and starboard side exhaust outlets are located below a water line of the boat.

10. The apparatus of claim 1, wherein the port side and starboard side exhaust outlets are located above a water line of the boat.

11. The apparatus of claim 10, wherein the port side and starboard side exhaust outlets extend vertically above the boat.

12. A method for directing a boat's engine exhaust out of either the port or starboard side of the boat, away from a person behind the boat to reduce exposure of the person to the engine exhaust, the boat having a engine with at least one exhaust manifold, a port side exhaust outlet connected by a first exhaust conduit to the at least one engine exhaust manifold of the boat, and a starboard side exhaust outlet connected by a second exhaust conduit to the at least one engine exhaust manifold of the boat, the method comprising:
controlling a flow of engine exhaust to permit the flow of engine exhaust through one of the first and second exhaust conduits, while preventing the flow of engine exhaust through the other of the first and second exhaust conduits, such that the engine exhaust is directed out of one of the port and starboard side exhaust outlets and not through the other of the port and starboard side exhaust outlets.

13. The method of claim 12, wherein said flow of engine exhaust to said port and starboard exhaust outlets is controlled by first and second valves in said first and second exhaust conduits, respectively, and said step of controlling flow of engine exhaust comprises opening one of the first and second valves and closing the other of the first and second valves, to direct the engine exhaust out of one of the port and starboard side exhaust outlets.

14. The method of claim 12, wherein the engine exhaust is directed out of either the port or starboard side of the boat below a water line of the boat.

15. The method of claim 12, wherein the engine exhaust is directed out of either the port or starboard side of the boat above a water line of the boat.

16. An apparatus for directing a power boat's engine exhaust away from a wake wave on which a person is wake surfing behind and to one of the port and starboard sides of the power boat to reduce exposure of the person to the engine exhaust, the power boat having an engine with at least one exhaust manifold, the apparatus comprising:
a port side exhaust outlet connected by a first exhaust conduit to the at least one engine exhaust manifold of the power boat;
a starboard side exhaust outlet connected by a second exhaust conduit to the at least one engine exhaust manifold of the power boat; and
valve means connected to said first and second exhaust conduits for directing the engine exhaust out of one of the port and starboard exhaust outlets.

17. The apparatus of claim 16, wherein said first and second exhaust conduits are connected to the at least one engine exhaust manifold at about a 90 degree angle.

18. The apparatus of claim 16, wherein said valve means comprises a first valve connected to the first exhaust conduit for controlling flow of engine exhaust through the first exhaust conduit to the port side exhaust outlet, and a second valve connected to the second exhaust conduit for controlling flow of engine exhaust through the second exhaust conduit to the starboard side exhaust outlet, and means for linking said first and second valves so that opening of one of the first and second valves closes the other of the first and second valves, such that the engine exhaust is directed out of one of the port or starboard side exhaust outlets.

19. The apparatus of claim 18, wherein said first and second valves are electrically controlled valves, and the means for linking the first and second valves comprises an electrical switch for opening one of said first and second valves and closing the other of said first and second valves.

20. The apparatus of claim 18, wherein said means for linking comprises a mechanical linkage between said first and second valves.

21. The apparatus of claim 16, wherein the valve means comprises a first valve connected to the first exhaust conduit for controlling flow of engine exhaust through the first exhaust conduit to the port side exhaust outlet, and a second valve connected to the second exhaust conduit for controlling flow of engine exhaust through the second exhaust conduit to the starboard side exhaust outlet, and means for linking said first and second valves so that opening of one of the first and second valves closes the other of the first and second valves, such that the engine exhaust is directed out of one of the port or starboard side exhaust outlets, and further comprising valve control means for controlling the valve means to switch the flow of engine exhaust from one of said port and starboard side exhaust outlets to the other of the port and starboard side exhaust outlets, and further comprising valve control means for controlling the valve means to switch the flow of engine exhaust from one of said port and starboard side exhaust outlets to the other of the port and starboard side exhaust outlets.

22. The apparatus of claim 21, wherein the means for linking comprises a mechanical linkage between said first and second valves, and said valve control means comprises a mechanical control connected to said mechanical linkage for opening one of said first and second valves and closing the other of said first and second valves.

23. The apparatus of claim 16, wherein the valve means is located outside the boat.

24. The apparatus of claim 16, wherein the port side and starboard side exhaust outlets are located below a water line of the boat.

25. The apparatus of claim 16, wherein the port side and starboard side exhaust outlets are located above a water line of the boat.

26. The apparatus of claim 25, wherein the port side and starboard side exhaust outlets extend vertically above the boat.

27. A method for directing a power boat's engine exhaust out of either the port or starboard side of the power boat, away from a wake wave on which a person is wake surfing behind the power boat and to one of the port and starboard sides of the power boat to reduce exposure of the person to the engine exhaust, the power boat having an engine with at least one exhaust manifold, the boat having a engine with at least one exhaust manifold, a port side exhaust outlet connected by a first exhaust conduit to the at least one engine exhaust manifold of the boat, and a starboard side exhaust outlet connected by a second exhaust conduit to the at least one engine exhaust manifold of the boat, the method comprising:
controlling flow of engine exhaust to permit the flow of engine exhaust through one of the first and second exhaust conduits, while preventing the flow of engine exhaust through the other of the first and second exhaust conduits, such that the engine exhaust is directed out of one of the port or starboard side exhaust outlets and not through the other of the port and starboard side exhaust outlets.

28. The method of claim 27, wherein said port and starboard exhaust outlets are connected to said at least one exhaust manifold by first and second valves, respectively, and said step of controlling flow of engine exhaust comprises opening one of the first and second valves and closing the other of the first and second valves, to direct the engine exhaust out of one of the port or starboard side exhaust outlets.

29. The method of claim 27, wherein the engine exhaust is directed out of either the port or starboard side of the boat below a water line of the boat.

30. The method of claim 27, wherein the engine exhaust is directed out of either the port or starboard side of the boat above a water line of the boat.

31. An apparatus for directing an engine exhaust away from the area in the wake of a boat, the engine having at least one exhaust manifold and an exhaust system directing exhaust gasses out of the rear of the boat, the apparatus comprising:
a secondary exhaust system arranged to direct the exhaust above the boat in a vertical direction away from the wake of the boat;
a valve in the exhaust system capable of diverting exhaust from the exhaust system into the secondary exhaust system; and
a control mechanism to actuate the valve to thereby direct exhaust gasses into the secondary exhaust system.

32. The apparatus of claim 31, further comprising:
a remote control for said control mechanism to allow an operator to divert said exhaust gasses into said secondary.

33. The apparatus of claim 32, wherein said remote control is a cable connected at one end to said valve and at the other end to a lever in said boat.

34. The apparatus of claim 32, wherein said valve is actuated by an electric motor and said remote control is a switch which actuates said motor.
